(19) United States
(12) Patent Application Publication
(10) Pub. No.: US 2010/0279389 A1
(43) Pub. Date: Nov. 4, 2010

(54) Modular algae culturing system and method

(76) Inventor: David Ziller, Pueblo, CO (US)

Correspondence Address: David Ziller, 772 Knox Drive, Pueblo, CO, 81007, US

(21) Appl. No.: 12/387,260

(22) Filed: Apr. 30, 2009

(51) Int. Cl. C12N 1/12; C12M 1/02

(52) U.S. Cl. 435/257.1; 435/303.3

(57) ABSTRACT

A modular algae culturing system and method are described. The system utilizes modular, vertically-oriented growth units that can be connected together in series or parallel (or any combination thereof). Each unit comprises: a back plate opposite a front plate, an input side wall opposite an output side wall, and a bottom plate opposite a top plate. The front plate is removable for cleaning, maintenance, etc. Furthermore, individual units are modular so they can be easily removed, repaired, replaced, etc. as needed. The unit is designed to hold a fluid mixture which generally comprises water, nutrients, and algae. Inside each unit is a plurality of generally horizontal mixing members which facilitate mixing of the mixture as it flows through the unit. The mixture flows from one or more processing stations through one or units. When mature, the algae can be harvested.

### TECHNICAL FIELD

The invention relates generally to culturing and harvesting of algae and more particularly to a modular system for culturing algae and a method of culturing algae using the system.

### BACKGROUND

As conventional hydrocarbon fuel sources are exhausted and the deleterious environmental effects of burning such fuels become better understood, a growing need for alternative fuel sources has been steadily building. To serve that need, the world has seen explosive growth in the biodiesel and ethanol industries. However, conventional crops can't produce enough of these biofuels per acre to meet the enormous demand. Furthermore, converting food production land and resources to produce biofuels causes reductions in the food supply and corresponding increases in the prices thereof. A potential solution exists in the form of lowly, simple algae.

Algae includes all microalgae, cyanobacteria and other similar organisms that act as very simple plants (often being single-celled), which includes over 40,000 known species. Like plants, algae utilize sunlight (or artificial light) to convert water and carbon dioxide into organic compounds through the process of photosynthesis. Furthermore, significant quantities of biodiesel, ethanol and even hydrogen can be produced from algae. Besides providing these potential fuels, algae can also be used to supplement human and animal food supplies and have other known and yet to be discovered uses in the fields of genetics, pharmaceuticals, agriculture, etc. A need therefore exists for a system to simply, quickly, and economically produce algae in order to harvest these resources from the algae.

A number of existing devices and methods for algae production already exist. For examples of devices and methods see U.S. Pat. No. 6,156,561 to Kodo; U.S. Pat. No. 5,981,271 to Doucha; U.S. Pat. No. 3,768,200 to Klock; U.S. Pat. No. 3,468,057 to Buisson; U.S. Pat. No. 6,037,170 to Sekine; U.S. Pat. No. 4,320,594 to Raymond; U.S. Pat. No. 4,217,728 to Shimanatsu; and U.S. Pat. No. 3,959,923 to Selke as well as international references including WO/2003/006629 to Sekine and WO/2007/098150 to Hu. There are many inefficiencies and problems associated with these existing devices and methods for the production of algae. Since many existing devices are open to the environment, they are continuously bombarded by external organisms (which compete with the algae for resources), inclement weather, and other threats to the efficient growth of the chosen algae strain(s). Additionally, any method or device that utilizes horizontally oriented growth units (i.e., pools, raceways, tanks, etc.) requires a significant amount of land-space or area for each growth unit. Other problems include low productivity due to a lack of environmental control (e.g., temperature swings), decreased penetration of light (i.e., the pools are too deep causing insufficient light to reach the algae in the deeper layers), and poor mixing causing nutrients to not be evenly distributed and thus retarding optimum algae growth.

A few existing devices address some of these problems by using closed or partially-closed growth systems that have a controlled environment and are oriented vertically. For example, in the '150 application to Hu, a partially-closed growth system using vertically oriented compartments is disclosed. However, the Hu system is constructed at the growth site and can only be extended by adding longer segments to existing compartments or rotating additional segments 90 degrees relative to existing compartments to form a serpentine shape.

The Hu system is not sufficiently flexible to fully take advantage of growth sites having varying terrain, limited space, etc. because it is constructed as very large, long containers instead of small, discrete units. Furthermore, once the Hu system is constructed, it is difficult to reconfigure as the components (sidewalls and struts) are bonded to each other using glue, epoxy, silicone, etc. For that same reason, it is also difficult to service, clean, repair, etc.

The Hu containers do not have horizontal or slightly sloping mixing members. Instead, Hu relies on aeration gas for mixing. Hu also uses “baffles” which are placed between the sidewalls of the Hu container and are oriented vertically to form many compartments within the overall container. These baffles extend 60%+ of the way up the sidewall of the Hu container so that the tops of all resulting compartments are in fluid communication. The bottoms of the Hu compartments can have a valve system connecting them, so normally they are in fluid communication, but can be closed-off from one another in case of breakage, etc. to ensure minimal loss of fluid. Hu baffles are placed 10 to 50 meters apart, causing them to function only as a means of reducing the overall container size into more manageable sub-containers, and not to facilitate mixing. When a portion of the Hu device breaks, a large amount of algae can be lost. Furthermore, the system can not flow while awaiting repairs as all fluid transfer between compartments must stop while the offending portion of the Hu device is repaired and again made water-tight.

None of the above mentioned prior art describes the unique features, objects, advantages and functions of the Modular Algae Culturing System and Method as described herein.

### SUMMARY

Embodiments described and claimed herein address the foregoing problems by providing a modular algae culturing system and method for quickly, easily, and efficiently culturing algae.

The system utilizes modular, vertically-oriented growth units that can be connected together in series or parallel (or any combination thereof). Each unit comprises: a back plate opposite a front plate, an input side wall opposite an output side wall, and a bottom plate opposite a top plate (collectively, components). The unit is designed to hold a fluid mixture which generally comprises water, nutrients, and algae. The components can be made of any light-transmitting material such as transparent or translucent plastic, acrylic, etc. In another embodiment, not all of the components are transparent, but preferably either or both of the front plate and back plate should allow light to enter the unit.

Each unit also has at least one input port and one output port. At least one of the input ports should be located near the top of the input side wall but could alternatively be located on the back or top plate in proximity thereto. At least one of the output ports should be located near the bottom of the output side wall but could alternatively be located on the back or bottom plate in proximity thereto. In another embodiment, the at least one input and/or the at least one output could be located on the front plate in proximity to the above disclosed locations; but this arrangement is less preferred.

Located between the front plate and the back plate in each unit are a plurality of mixing members. Each member is oriented generally parallel with the top and bottom plates. The first mixing member extends from the input side wall towards the output side wall, stopping short of contacting the output side wall. It is preferable that each mixing member contact both the front plate and back plate. The second mixing member extends from the output side wall towards the input side wall, stopping short of contacting the input side wall. Subsequent mixing members alternate as they move down the unit ending with a mixing member extending from the output side wall. In an alternate embodiment, at least one output is on the same side wall as at least one input and so the final mixing member would extend from the wall opposite the side wall containing the output. Thus, it is possible to utilize only one mixing member, but it is preferable to have 4 or more, depending on the overall dimensions of the unit, the rate of flow of the mixture, environmental factors such as temperature and amount of light, etc.

The mixing members facilitate proper mixing of the fluid, nutrient, and algae mixture to maximize efficient algae growth. With proper mixing, dead spots where algae growth is inhibited are minimized. Furthermore, the algae cells tend not to clump together and lodge on a unit's components or mixing members if the mixture is kept sufficiently mixed. Other methods of mixing used in the art include: mechanical means that waste energy, are inefficient mixers, and can damage algae cells; and aeration that is inefficient and costly. The mixing members facilitate proper circulation of the algae through the unit so that the mixture travels uniformly at the appropriate rate. Without mixing members, portions of the mixture are exposed to the light for non-efficient time periods. When exposed for too long a period, the mixture can become too hot and build unwanted pressure in the system. When exposed for too short a period, the mixture doesn't allow for efficient growth of the algae.

The system uses a plurality of these units to culture algae. The units are constructed so as to easily and interchangeably attach to one another. Thus, as the needs of a particular installation of the growth system change, the number and arrangement of units can be varied accordingly. If any particular unit becomes damaged or otherwise needs to be replaced, it is simply removed from the system and a new modular unit is installed in its place. In addition, if the location of the growth system has varying terrain and/or features that limit the arrangement, shape, and overall size of the growth system, more or fewer modular units can be attached to the system to accommodate the given area.

The front plate of each unit is easily removable to assist in cleaning, maintenance, etc. This is an important feature as a key problem with many existing algae growth systems is difficult access for cleaning and maintenance that increases costs and decreases growth efficiency.

The growth units constitute a closed-type system, thus significantly reducing competition from foreign organisms, reduction in algae production (and even physical damage) caused by inclement weather, and other uncontrolled environmental impacts. Furthermore, the individual units can be removed, replaced, repaired, and cleaned without disrupting the entire system simply by turning one or more valves (or similar structures) and redirecting the mixture arriving from the input pipe(s) directly to the output pipe(s). The unit in question can then be drained and removed, cleaned, serviced, etc. while the mixture continues to flow throughout the remaining units and the rest of the system.

In one embodiment, a plurality of growth units are attached together and the fluid mixture containing the algae is allowed to circulate between them. In a series connection, the mixture enters the first unit through the one or more inputs. The mixture then travels generally horizontally until the first mixing member ends; the mixture then tumbles down and thoroughly mixes before meeting the next mixing member and flowing in the other direction. At the end of each mixing member, the fluid swirls as it tumbles downwards causing chaotic vortices and/or turbulence that facilitate the mixing. Once the mixture has reached the bottom of the unit, it exits from at least one output and is then routed to at least one input on the next unit. The process continues until the final unit is traversed at which time the mixture is then directed to one or more processing stations (or returned directly to one or more of the units). In a parallel connection, the mixture is directed to a manifold which splits the mixture into multiple pipes each leading to a single unit. The mixture from a given pipe traverses one unit and is then brought back together to one or more common processing stations with the mixtures from the other pipes. In other embodiments, combinations of parallel and series connections are contemplated.

At the one or more processing stations, the inbound mixture is routed into a main feed tank where additional nutrients, fluid, etc. can be added. The mixture is then routed from the feed tank to a pump, or other means of moving the mixture, and through a gate or valve that can direct the mixture either into a harvest tank for removal of algae or back out to the plurality of growth units. Additional components can be added in the processing station(s) without departing from the scope of this invention.

Once the algae have been exposed to light for a sufficient period of time to mature, the algae can be harvested (alternatively, the algae can be removed from the system at any time). This is accomplished by switching a gate, valve, etc. at a processing station to redirect the flow of the mixture into the harvest tank(s).

Another aspect of the invention provides methods comprising one or more of the following steps: setting up a modular algae culturing system using a plurality of units, introducing a mixture, introducing algae, operating the system, bypassing units as desired, and harvesting algae.

These and other objects of the present invention will become apparent to those familiar with different types of algae culturing devices and methods when reviewing the following detailed description, showing novel construction, combination, and elements as described herein, and more particularly as defined by the claims; it being understood that variations in the embodiments to the herein disclosed invention are meant to be included within the scope of the claims, except insofar as they may be precluded by the prior art.

### BRIEF DESCRIPTION OF THE DRAWINGS

The aforementioned and other features and objects of the present invention and the manner of attaining them will become more apparent and the invention itself will be best understood by reference to the following description of a preferred embodiment and other embodiments taken in conjunction with the accompanying drawings, wherein:

FIG. 1 illustrates a perspective view of an exemplary embodiment of a single unit of a modular algae culturing system.

FIG. 2 illustrates a front view of an exemplary embodiment of a single unit of a modular algae culturing system highlighting the flow-path of a mixture within the unit.

FIG. 3 illustrates a left side view of an exemplary embodiment of a single unit of a modular algae culturing system wherein the left side is the input side.

FIG. 4 illustrates a right side view of an exemplary embodiment of a single unit of a modular algae culturing system wherein the right side is the output side.

FIG. 5 illustrates a top view of an exemplary embodiment of a modular algae culturing system arranged in a pie configuration.

FIG. 6 illustrates exemplary operations for a method of culturing algae using a modular algae culturing system.

### DETAILED DESCRIPTION

In one embodiment, the system utilizes one or more vertically-oriented growth units that can be connected together. Each unit comprises: input/output ports, at least one mixing member, a back plate substantially opposite a front plate, an input side wall substantially opposite an output side wall, and a bottom plate substantially opposite a top plate (collectively, components). The unit is designed to hold a fluid mixture which generally comprises water, nutrients, and algae. It is contemplated that the system could be used for culturing other organisms besides algae. The components can be made of any light-transmitting material such as transparent or translucent plastic, acrylic, etc. In another embodiment, not all of the components are transparent, but preferably either or both of the front plate and back plate should allow light to enter the unit. Varying degrees of transparency or translucency can be utilized; however it is generally preferable to use components that transmit the maximum amount of light possible without being overly expensive.

The system uses a plurality of units to culture algae. The units are constructed so as to easily and interchangeably attach to one another. Thus, as the needs of a particular installation of the growth system change, the number and arrangement of units can be varied accordingly. If any particular unit becomes damaged or otherwise needs to be replaced, it is simply removed from the system and a new modular unit is installed in its place.

It is preferable that the light source is natural (i.e., the sun); however, artificial light source(s) could be used. Substantial additional costs can be associated with artificial lighting and such lighting can make the overall system overly complicated and unwieldy. Nevertheless, artificial light(s) can be incorporated either internally or externally to the individual unit.

Construction of a unit can be accomplished in a variety of ways. In one embodiment, plastic welding means are used to adhere the back plate to each of the input side wall, the output side wall, the bottom plate and the top plate. Additionally, the one or more mixing members can be welded to the back plate as well. Other means of attaching the components to each other are contemplated and can be utilized. However, regardless of the means used, the front plate should be not be permanently attached to the unit as it should remain removable for ease of access to the inside of the unit. The front plate can be attached via a hinge or by other means on one of its edges, but it should be otherwise allowed to open away from the remaining components. Easy access to the interior of the unit is an important feature as a key problem with many existing algae growth systems is difficult access for cleaning and maintenance. Such difficulties can increase costs and decrease growth efficiency.

Each unit has at least one input port and one output port. It is preferable that the input(s) are located in proximity to the top of the unit and the output(s) are located in proximity to the bottom of the unit as gravity can then assist in the resulting flow of the mixture from the input(s) to the output(s). At least one of the inputs should be located near the top of the input side wall but could alternatively be located on the back plate or top plate in proximity thereto. At least one of the outputs should be located near the bottom of the output side wall but could alternatively be located on the back plate or bottom plate in proximity thereto. In another embodiment, the at least one input and/or the at least one output could be located on the front plate in proximity to the above disclosed locations; but this arrangement is less preferred. In yet another embodiment, the ports are located elsewhere.

Located between the front plate and the back plate in each unit are a plurality of mixing members. Each member is oriented generally parallel with the top and bottom plates and generally perpendicular to the input and output side walls. The mixing members can have a slightly downwards sloping orientation, but it is not preferable for them to slope upwards. The mixing members generally have a rectangular cross-section, but could also have a triangular cross section where the base of the triangle attaches to the side wall. The first mixing member extends from the input side wall towards the output side wall, stopping short of contacting the output side wall. The distance between the output side wall and the first member can vary, but it is preferably similar to the distance between the front plate and the back plate. It is preferable that each mixing member contact both the front plate and the back plate. A second mixing member can extend from the output side wall towards the input side wall, stopping short of contacting the input side wall. The distance between the output side wall and the first member can vary, but it is preferably similar to the distance between the front plate and the back plate. Any subsequent mixing members alternate as they move down the unit; in one embodiment, ending with a mixing member extending from the output side wall. In an alternate embodiment, at least one output is on the same side wall as at least one input and so the final mixing member would extend from the wall opposite the side wall containing the output. Thus, it is possible to utilize only one mixing member, but it is preferable to have 4 or more, depending on the overall dimensions of the unit, the rate of flow of the mixture, environmental factors such as temperature and amount of light, etc.

The mixing members facilitate proper mixing of the fluid(s), nutrient(s), and algae mixture to maximize efficient algae growth. With proper mixing, dead spots where algae growth is inhibited are minimized. Furthermore, the algae cells tend not to clump together and lodge on a unit's components if the mixture is kept sufficiently mixed. Other methods of mixing used in the art include: mechanical means that waste energy, are inefficient mixers, and can damage algae cells; and aeration that is inefficient and costly. Such mixing means, although not preferable, could also be used in addition to the mixing members. The mixing members facilitate proper circulation of the algae through the unit so that the mixture travels uniformly at the appropriate rate. Without mixing members, portions of the mixture are exposed to the light source for non-efficient time periods. When exposed for too long a period, the mixture can become too hot and build unwanted pressure in the system and can reduce efficiency. When exposed for too short a period, the amount of light received by the algae does not allow for efficient growth of the algae.

The growth units constitute a closed-type system, thus significantly reducing competition from foreign organisms, reduction in algae production (and even physical damage) caused by inclement weather, and other uncontrolled environmental impacts. For example, in open-pool algae growth systems, heavy rains or floods can wash-out the pools, remove or kill the algae and even destroy the entire system. Furthermore, competing algae or other foreign organisms can enter such open systems quite easily from a variety of vectors. Such competition can reduce the efficiency of the system and even cause the desired algae to die out. Thus, it is preferable to have a closed-type system.

The individual units can be removed, replaced, repaired, and/or cleaned without disrupting the entire system simply by turning one or more valves (or similar structures) and redirecting the mixture arriving from the input pipe(s) directly to the output pipe(s). The unit in question can then be drained and removed, cleaned, serviced, etc. Further, since the front plate is easily opened, relatively quick cleaning or maintenance can be performed without removing the entire unit from its location. In order to accomplish the draining of a unit, a portable pump could be used. Alternate means are contemplated, including the use of gravity to assist in emptying the unit into a temporary holding container.

In one embodiment, a plurality of growth units are attached together and a fluid mixture containing the algae is allowed to circulate between and through them. In a simple, inexpensive configuration, individual units in a system are connected directly one to the next with the output port(s) from one unit connecting to the input port(s) from the next unit, and so on. After the mixture leaves the final unit, it is routed to a processing station where it flows through a feed tank, a pump (or other means of moving the mixture), and then back out to the first unit to complete another circuit. Other embodiments can include more complex extra-unit flow patterns.

In one possible series connection embodiment, the mixture enters the first unit through the one or more inputs. The mixture then travels generally horizontally until the first mixing member ends; the mixture then tumbles down and mixes before meeting the next mixing member and flowing in the other direction. At the end of each mixing member, the fluid swirls as it tumbles downwards causing chaotic vortices and/or turbulence that facilitate the mixing. Once the mixture has reached the bottom of the unit, it exits from at least one output and is then routed to at least one input on the next unit. The process continues until the final unit is traversed at which time the mixture is then directed to one or more processing stations (or returned directly to one or more of the units).

In one possible parallel connection embodiment, the mixture is directed to a manifold which splits the mixture into multiple pipes each leading to a single unit. The mixture from each pipe traverses one unit and is then brought back together to one or more common processing stations with the mixtures from other pipes. In other embodiments, combinations of parallel and series connections are contemplated.

At the one or more processing stations, the inbound mixture can be routed into a main feed tank where additional nutrients, fluids, etc. can be added. The main feed tank can contain aeration equipment, sensors, and other components known in the art which facilitate algae culturing and further enhance the efficiency of the system. Once the mixture has left the feed tank, it can then be routed to a pump, or other means of moving the mixture, and through a gate or valve that can direct the mixture either into a harvest tank for removal of algae or back out to the plurality of growth units. Additional components can be added in the processing station(s) without departing from the scope of this invention.

In one embodiment, once the algae have been exposed to light, nutrients, etc. for a sufficient period of time to substantially mature, the algae can be harvested. In another embodiment, the algae can be harvested or otherwise removed from the system at any time. Harvesting or removal is accomplished by switching a gate, valve, etc. at a processing station to redirect the flow of the mixture into the harvest tank(s). In other embodiments, other means of effecting removal can be utilized.

Another aspect of the invention provides methods for culturing algae using a modular algae culturing system comprising one or more of the following steps: setting up a modular algae culturing system using a plurality of units, introducing a mixture, introducing algae, operating the system, bypassing units as desired, and harvesting algae.

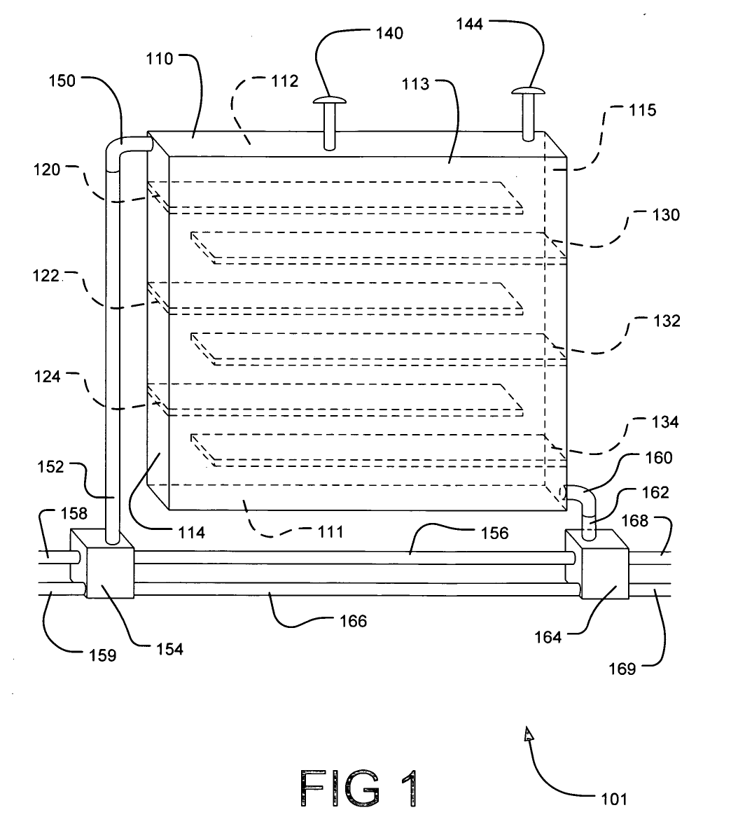

FIG. 1 illustrates a perspective view of an exemplary embodiment of a single unit 101 of a modular algae culturing system. In the embodiment shown in FIG. 1, a unit 101 has main components as follows: a top plate 110 substantially opposite a bottom plate 111; a back plate 112 substantially opposite a front plate 113; an input side wall 114 substantially opposite an output side wall 115; a plurality of input side mixing members 120, 122, and 124 in proximity to the input side wall 114; a plurality of output side mixing members 130, 132, and 134 in proximity to the output side wall 115; an input port 150; and an output port 160. Additional components include a first vent 140; a second vent 144; an input pipe 152; an output pipe 162; an input box 154; an output box 164; an incoming pipe 158, a return pipe 159, an outgoing pipe 168, a station-bound pipe 169, a unit bypass pipe 156; and a transmit pipe 166. The unit 101 is designed to hold a fluid mixture (not shown in FIG. 1 but generally located in the interior of the unit 101 defined as the volume between the plates 110, 111, 112, and 113; walls 114 and 115; and mixing members 120, 122, 124, 130, 132, and 134) which generally comprises water, nutrients, and algae. The use of the terms “plates” and “walls” as used herein should in no way limit the shape of the described objects to only rectangular, flat shapes; although a rectangular cuboid shape for the unit is preferable, it could also be ovaloid, cylindrical, or any other shape that allows for the same basic functionality.

Construction of a unit 101 can be accomplished in a variety of ways. In one embodiment, plastic welding means are used to adhere the back plate 112 to each of the input side wall 114, the output side wall 115, the bottom plate 111 and the top plate 110. Each of the plates 110, 111, 112, and 113 and walls 114 and 115 are generally rectangular in shape. Additionally, the one or more mixing members 120, 122, 124, 130, 132, and 134 are generally rectangular in shape as well. Although, as mentioned above, the mixing members can have a generally rectangular or triangular cross section. Plastic welding means can be used to adhere the mixing members 120, 122, 124, 130, 132, and 134 to the back plate 112. Other means of attaching the components to each other are contemplated and can be utilized. However, regardless of the means used, the front plate 113 should be not be permanently attached to the unit 101 as it should remain removable for ease of access to the inside of the unit 101. The front plate 113 can be attached via a hinge or by other means on one of its edges, but it should otherwise be allowed to open away from the remaining components. Easy access to the interior of the unit 101 is an important feature as a key problem with many existing algae growth systems is difficult access for cleaning and maintenance. Such difficulties can increase costs and decrease growth efficiency.

As can be seen in FIG. 1, the plates 110, 111, 112, and 113 and the walls 114 and 115 are brought together to form a six-sided rectangular cuboid (or box). In other embodiments, other shapes are contemplated. For example, the top plate, output side plate, bottom plate and input side plate could form an oval or circular shape. Inside the cuboid main body of the unit 101 are the mixing members 120, 122, 124, 130, 132, and 134.

Each unit 101 has at least one input port 150 and one output port 160. It is preferable that the input(s) are located in proximity to the top plate 110 of the unit 101 and the output(s) are located in proximity to the bottom plate 111 of the unit 101 as gravity can then assist in the resulting flow of the mixture from the input(s) to the output(s). At least one of the input ports 150 should be located near the top of the input side wall 114 but could alternatively be located on the back plate 112 or the top plate 110 in proximity thereto. At least one of the output ports 160 should be located near the bottom of the output side wall 115 but could alternatively be located on the back plate 112 or bottom plate 111 in proximity thereto. In another embodiment, the at least one input port 150 and/or the at least one output port 160 could be located on the front plate 113 in proximity to the above disclosed locations; but this arrangement is less preferred. In yet another embodiment, the ports 150 and 160 are located elsewhere.

Located between the front plate 113 and the back plate 112 in each unit 101 are a plurality of mixing members 120, 122, 124, 130, 132, and 134. Each member is oriented generally parallel with the top and bottom plates 110 and 111 and generally perpendicular to the input and output side walls 114 and 115. Although the mixing members 120, 122, 124, 130, 132, and 134 can be oriented at a sloping angle (as opposed to exactly parallel with the top plate and/or bottom plate) as well. It is not preferable for the members to be oriented with an upwards sloping angle. In the embodiment illustrated in FIG. 1, the first mixing member 120 extends from the input side wall 114 towards the output side wall 115, stopping short of contacting the output side wall 115. The distance between the output side wall 115 and the first member 120 can vary, but it is preferably similar to the distance between the front plate 113 and the back plate 112. It is preferable that each mixing member 120, 122, 124, 130, 132, and 134 contact both the front plate 113 and back plate 112. A second mixing member 130 can extend from the output side wall 115 towards the input side wall 114, stopping short of contacting the input side wall 114. The distance between the input side wall 114 and the second member 130 can vary, but it is preferably similar to the distance between the front plate 113 and the back plate 112. Any subsequent mixing members alternate as they move down the interior of the unit 101. In one embodiment, the progression ends with a mixing member 134 extending from the output side wall 115. In an alternate embodiment, at least one output port 160 is on the same side wall as at least one input port 150 and so the final mixing member can extend from the wall opposite the side wall containing the output. Thus, it is possible to utilize only one mixing member, but it is preferable to have 4 or more, depending on the overall dimensions of the unit, the rate of flow of the mixture, environmental factors such as temperature and amount of available light, etc.

The mixing members 120, 122, 124, 130, 132, and 134 facilitate proper mixing of the fluid, nutrient, and algae mixture to maximize efficient algae growth. With proper mixing, dead spots where algae growth is inhibited are minimized. Furthermore, the algae cells tend not to clump together and lodge on a unit's components if the mixture is kept sufficiently mixed. The mixing members 120, 122, 124, 130, 132, and 134 facilitate proper circulation of the algae through the main body of the unit 101 so that the mixture travels uniformly at the appropriate rate.

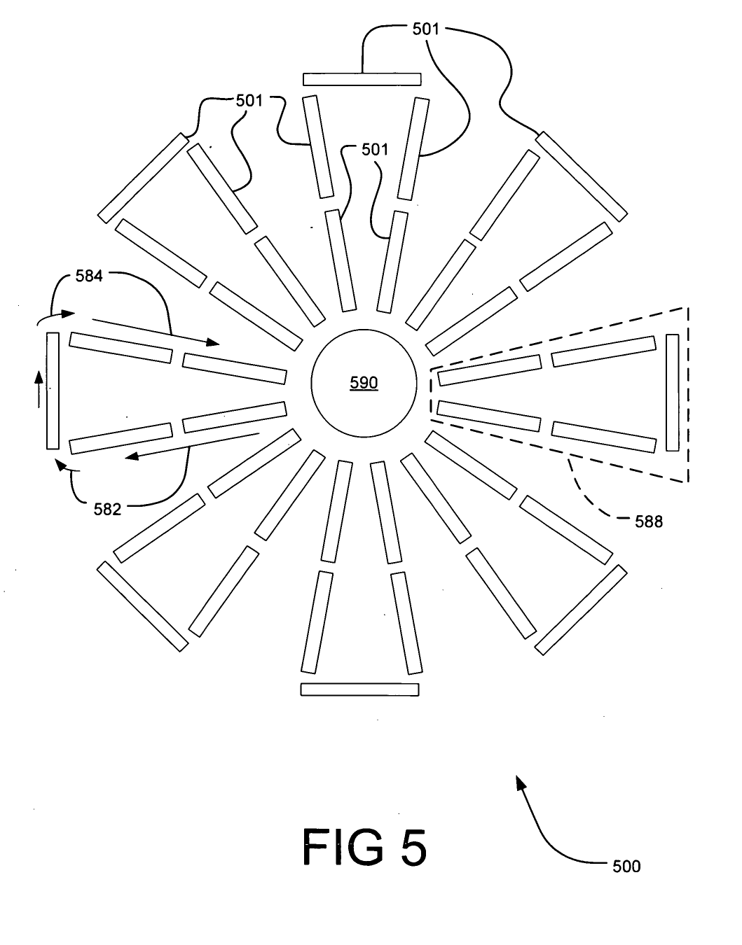

The mixture enters the single unit 101 in FIG. 1 at the input box 154. The input box 154 is a device capable of receiving an incoming pipe 158 carrying the mixture and it contains a number of valves or means of directing the flow of the mixture. The input box 154 is not necessarily box-shaped, but can be any shape. The incoming pipe 158 can come directly from the processing station (see FIG. 5) or can be attached to the outgoing pipe from the previous unit in the system (for a representation of the outgoing pipe from this unit, see FIG. 1, item 168). In the embodiment illustrated in FIG. 1, the input box 154 can have two valves (not shown in FIG. 1). The first valve is a three-way valve and is connected to the incoming pipe 158. The first valve receives the incoming mixture from the incoming pipe 158 and then directs it in one of three directions: in normal operation, the incoming mixture is directed into the input pipe 152 for routing into the unit 101; when the unit 101 needs to be cleaned, maintained, etc., the first valve directs the mixture onwards into the unit bypass pipe 156 so as to bypass the main body of the unit 101; and finally, when the mixture needs to be returned back to the processing station, the first valve directs the mixture into the second valve. The second valve can be set to receive the mixture from the first valve and direct it out of the input box 154 into a return pipe 159 back towards the processing station. Alternatively, the second valve can be set to receive the mixture flowing from the output box 164, through the transmit pipe 166, and direct it into the return pipe 159 back towards the processing station.

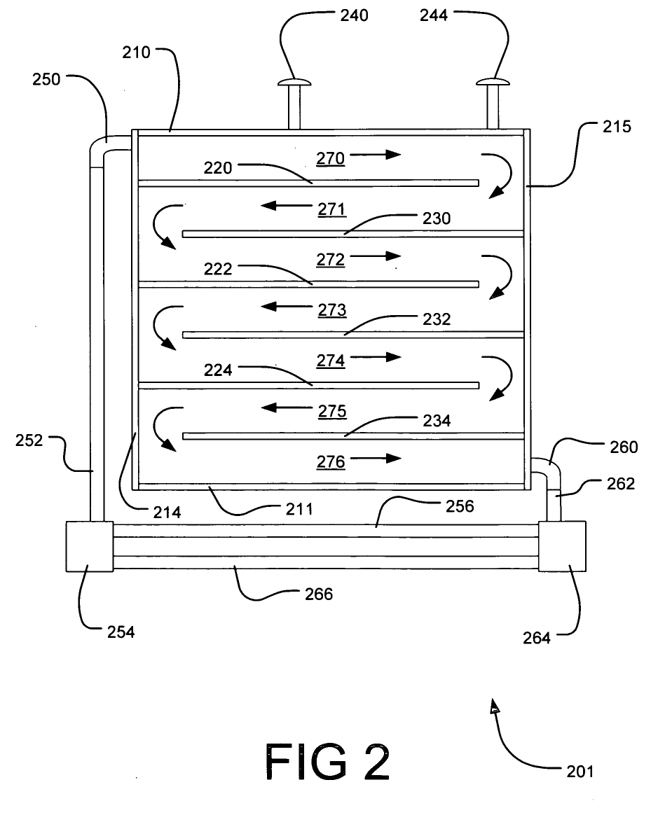

During normal operation, the mixture moves from the input box 154 into the input pipe 152. It flows through the input pipe 152 to the input port 150 and through the input port 150 into the main body of the unit 101. After traversing the main body of the unit 101 (see detailed description of FIG. 2), the mixture flows through the output port 160 into the output pipe 162 and then into the output box 164 where it can be redirected by a number of valves or other means of directing the flow of the mixture. The output box 164 is not necessarily box-shaped, but can be any shape. In the embodiment illustrated in FIG. 1, the output box 164 contains two valves (not shown in FIG. 1). The first valve receives the mixture from either the output pipe 162 (in normal operation) or the unit bypass pipe 156 (when the main body of the unit 101 is being bypassed). The first valve then directs the mixture into either the outgoing pipe 168 (if additional units are in the system) or to the second valve (if this unit is the final unit in the system). The second valve receives the mixture from either the first valve or from the station-bound pipe 169 and directs it into the transmit pipe 166. The transmit pipe 166 directs the mixture back into the input box 154 where it continues into the return pipe 159 and onwards towards the processing station (see FIG. 5). The station-bound pipe 169 receives the mixture from the return pipe of the prior unit in the system.

When the system is functioning, one or more gases can be produced. Furthermore, as light enters the unit 101, it can increase internal temperatures. As the volume of gas(es) increases and temperatures rise, the system can experience an increase in pressure. Thus, there is a need for vents 140 and 144. In the embodiment shown in FIG. 1, two vents 140 and 144 are illustrated. Other embodiments can contain more or fewer vents.

The overall modular algae culturing system uses a plurality of these modular units 101 to culture algae. The units 101 are constructed so as to easily and interchangeably attach to one another. Thus, as the needs of a particular installation of the growth system change, the number and arrangement of units 101 can be varied accordingly. If any particular unit 101 becomes damaged or otherwise needs to be replaced, it is simply isolated from the flow of the system and then removed and a new modular unit 101 is installed in its place.

FIG. 2 illustrates a front view of an exemplary embodiment of a single unit 201 of a modular algae culturing system highlighting the flow-path of a mixture within the unit 201. In the embodiment shown in FIG. 2, a unit 201 has main components as follows: a top plate 210 substantially opposite a bottom plate 211; (the back plate and front plate are not shown in FIG. 2, see FIG. 1, items 112 and 113, respectively) an input side wall 214 substantially opposite an output side wall 215; a plurality of input side mixing members 220, 222, and 224 in proximity to the input side wall 214; a plurality of output side mixing members 230, 232, and 234 in proximity to the output side wall 215; an input port 250; and an output port 260. Additional components include a first vent 240; a second vent 244; an input pipe 252; an output pipe 262; an input box 254; an output box 264; a unit bypass pipe 256; and a transmit pipe 266. The unit 201 is designed to hold a fluid mixture (not shown in FIG. 2 but generally located in the interior of the unit 201 defined as the volume between the plates, walls and mixing members) which generally comprises water, nutrients, and algae.

The mixture enters the single unit 201 in FIG. 2 at the input box 254. The input box 254 is a device capable of receiving an incoming pipe (see FIG. 1, item 158) carrying the mixture and it contains a number of valves. In the embodiment illustrated in FIG. 2, the input box 254 can have one valve (not shown in FIG. 2). The valve is a two-way valve and is connected to the incoming pipe. The valve receives the incoming mixture from the incoming pipe and then directs it in one of two directions. During normal operation, the incoming mixture is directed into the input pipe 252 for routing into the main body of the unit 201. When the unit 201 needs to be cleaned, maintained, etc., the valve directs the mixture onwards into the unit bypass pipe 256 so as to bypass the main body of the unit 201. The input box 254 can also act as a simple pass-through that receives the mixture flowing from the transmit pipe 266 and directs it into the return pipe (see FIG. 1, item 159) back towards the processing station. In yet another embodiment, the transmit pipe 266 is connected directly to the return pipe and does not pass-through the input box 254.

Assuming the unit 201 is under normal operation, the mixture travels from the input pipe 252 into the main body of the unit 201 through the input port 250. The mixture then flows horizontally along the first mixing member 220 from the input side wall 214 towards the output side wall 215; this flow is represented by the arrow 270. When the mixture reaches the output side wall 215, it is directed down towards the second mixing member 230. As the mixture falls over the end of the first mixing member 220 vortices and turbulence are created, facilitating further mixing of the mixture. The mixture then progresses horizontally along the second mixing member 230 towards the input side wall 214; this flow is represented by the arrow 271. This back and forth flow continues through the main body of the unit 201 (see flow arrows 272, 273, 274, 275, and 276), the mixture being further mixed each time the flow direction is reversed, until the mixture exits through the output port 260.

The mixture exits the single unit 201 in FIG. 2 at the output port 260. It then flows through the output pipe 262 and into the output box 264. In the embodiment illustrated in FIG. 2, the output box 264 can have one valve (not shown in FIG. 2). The valve is a two-way valve and is connected to the bypass pipe 256 and the output pipe 262. The valve receives the mixture from either the output pipe 262 (during normal operation) or from the bypass pipe 256 (when the main body of the unit 201 is being bypassed) and then directs it in onwards into an outgoing pipe (see FIG. 1, item 168). The output box 264 can also act as a simple pass-through that receives the mixture flowing from the station-bound pipe (see FIG. 1, item 269) and directs it into the transmit pipe 266. In an alternate embodiment, the station-bound pipe is connected directly to the transmit pipe and does not pass-through the output box 264. In yet another embodiment, the unit shown in FIG. 2 is the final unit in the system and the mixture does not enter an output box at all but is directed from the output pipe 262 back to the processing station (see FIG. 5).

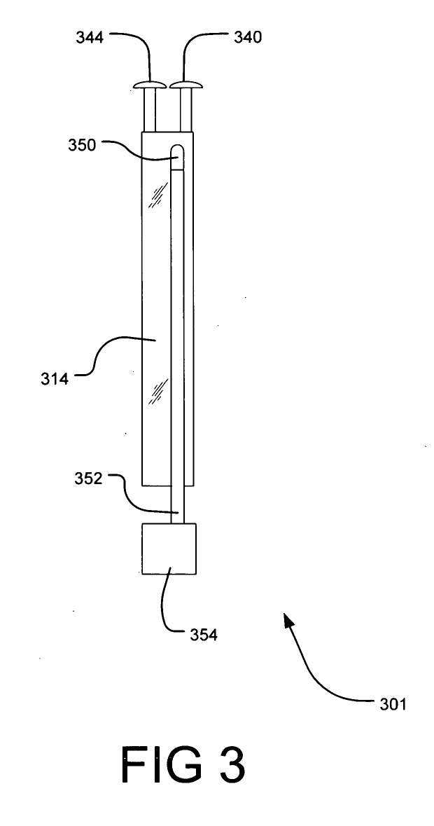

FIG. 3 illustrates a left side view of an exemplary embodiment of a single unit 301 of a modular algae culturing system wherein the left side is the input side. In the embodiment shown in FIG. 3, a unit 301 has the following illustrated components: an input side wall 314; an input port 350; a first vent 340; a second vent 344; an input pipe 352; and an input box 354.

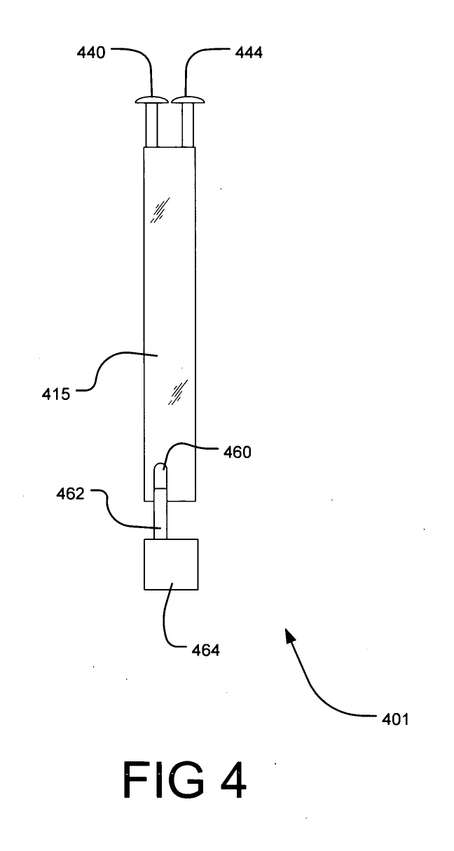

FIG. 4 illustrates a right side view of an exemplary embodiment of a single unit 401 of a modular algae culturing system wherein the right side is the output side. In the embodiment shown in FIG. 4, a unit 401 has the following illustrated components: an output side wall 415; an output port 460; a first vent 440; a second vent 444; an output pipe 462; and an output box 464.

FIG. 5 illustrates a top view of an exemplary embodiment of a modular algae culturing system 500 arranged in a pie configuration. In this view, only the top plates of the individual units 501 can be seen. For clarity, the pipes running between the individual units 501 are not illustrated in FIG. 5. An individual piece 588 in the pie comprises a number of units directed in a circuit outwards from the processing station 590 and then returning back to the processing station 590. This flow is represented by the flow arrows 582 and 584, wherein the outbound flow from the processing station 590 is represented by outbound arrows 582 and the inbound flow back towards the processing station 590 is represented by inbound arrows 584. This type of pie-shaped configuration has some significant advantages over other configurations as no transmit pipes, return pipes, or station-bound pipes need to be used.

In the embodiment shown in FIG. 5, only one processing station 590 is represented. A modular algae culturing system 500 can have more than one processing station 590. Processing stations are well known in the art and usually contain at least a main feed tank, a pump or other means of moving the mixture, and a harvest tank or other means of harvesting the algae. All inbound pipes returning from the plurality of growth units 501 contain the mixture and are routed into the main feed tank where additional nutrients, fluid, etc. can be added to the mixture. The mixture is then routed from the feed tank to a pump, or other means of moving the mixture, and through a gate or valve that can direct the mixture either into a harvest tank for removal of algae or back out to the plurality of growth units through a manifold or some other means of splitting the flow of mixture evenly to each pie piece 588. Once the algae have been exposed to light for a sufficient period of time to mature, the algae can be harvested (alternatively, the algae can be removed from the system 500 at any time). This is accomplished by switching a gate, valve, etc. at a processing station 590 to redirect the flow of the mixture into the harvest tank(s).

In an alternate embodiment, the pump is situated before the main feed tank. In yet other embodiments, the components residing in the processing station 590 are placed in other orders relative to each other. Additional components can be added in the processing station(s) 590 without departing from the scope of this invention.

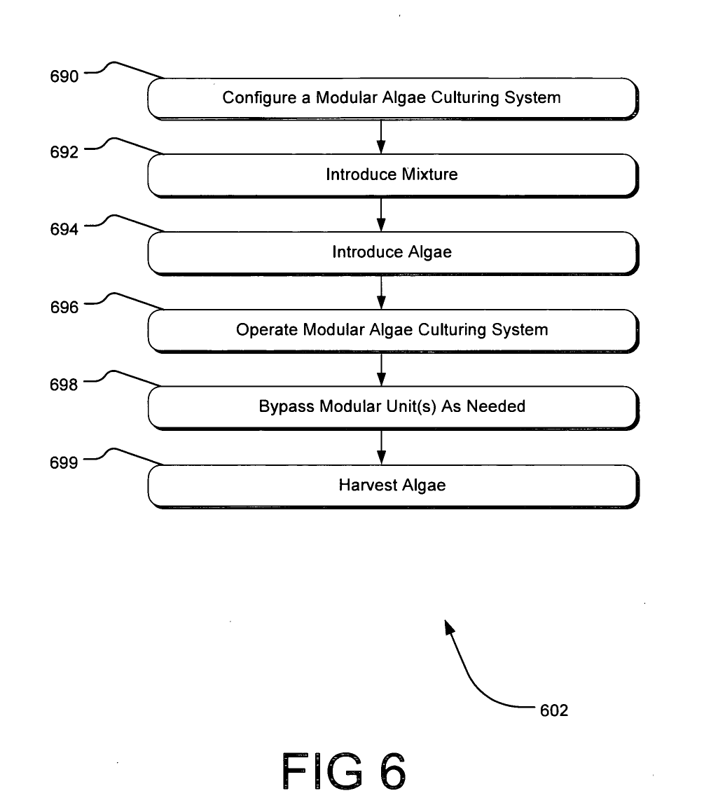

FIG. 6 illustrates exemplary operations 602 for a method of culturing algae using a modular algae culturing system. The Configure operation 690 involves connecting one or more units together with each other and with one or more processing stations. As discussed in detail above, individual modular units can be attached in series, parallel, or some combination thereof that allows the mixture to flow through them and be eventually routed back to one or more processing stations. For an example of a combined series and parallel system, see FIG. 5 where the individual piece of the pie 580 have units connected in series, while the multiple pie pieces 580 are connected in parallel to each other. Other methods of configuring the system are contemplated.

The Introduce Mixture operation 692 involves the introduction of a mixture of nutrients and water into the system. The introduction can be accomplished by adding water to the entire system and then gradually introducing nutrients through the main feed tank. In an alternate embodiment, the nutrients can be pre-mixed with the water and they can be introduced into the system simultaneously. Nutrients are not limited simply to food for the algae but could include any other item that is desired to be added to the mixture. Other methods of introducing the mixture are contemplated.

The Introduce Algae operation 694 involves the introduction of one or more types of algae into the system. The introduction can be accomplished by adding the algae throughout the entire system (using the one of more inputs at each unit) or the algae can be introduced relatively gradually into the system through the main feed tank. Other methods of introducing the algae are contemplated.

The Operate Modular Algae Culturing System operation 696 involves starting the system and allowing the mixture to flow. The individual units in the system are exposed to sunlight (and/or some artificial light source) causing the algae in the system to begin photosynthesis, grow and reproduce. Operating the System can include introducing additional nutrients, water, algae, etc. to the system. Other methods of operating the system are contemplated.

The Bypass Modular Unit(s) As Needed operation 698 involves using the valve systems (examples discussed above) or other means to redirect the flow of the mixture in the system around one or more units. The units in question can then be cleaned, maintained, or even removed from the system, as needed. The Bypass operation can also include returning the system to its earlier state by resetting the valves or redirect means to their original condition. Other methods of bypassing the units are contemplated.

The Harvest Algae operation 699 involves the removal of the algae from the system. Once the algae have been exposed to light for a sufficient period of time to mature, the algae can be harvested (alternatively, the algae can be removed from the system at any time). This is accomplished by switching a gate, valve, etc. at a processing station to redirect the flow of the mixture into the harvest tank(s). Known harvesting means that exist in the art, or are yet to be developed, can then be used to remove the algae from the system. Other methods of harvesting the algae are contemplated.

The above specification, examples and data provide a description of the structure and use of exemplary embodiments of the described articles of manufacture and methods. Many embodiments can be made without departing from the spirit and scope of the invention.

What is claimed is:

1. A modular algae culturing unit, comprising:
(i) a substantially watertight main body comprising:
(a) a top plate substantially opposite a bottom plate;
(b) a back plate substantially opposite a front plate;
(c) an input side wall substantially opposite an output side wall;
(d) wherein the top plate, the bottom plate, the back plate, the front plate, the input side wall and the output side wall are placed in proximity to one another so as to define an interior space and an exterior space;
(e) a plurality of mixing members within the interior space, the plurality of mixing members being oriented generally horizontally;
(f) an input port providing access from the exterior space into the interior space; and
(g) an output port providing access from the interior space out to the exterior space;
(ii) an input pipe wherein a mixture comprising at least a fluid can arrive from the input pipe and flow into the input port into the main body where the mixture can flow through the interior space and be mixed by the plurality of mixing members; and
(iii) an output pipe wherein the mixture can flow out of the interior space via the output port and into the output pipe.

2. The unit of claim 1 further comprising an input box having a plurality of valves for directing flows of the mixture.

3. The unit of claim 1 further comprising an output box having a plurality of valves for directing flows of the mixture.

4. The unit of claim 2 wherein the input box has a first input valve that can accept the mixture from an incoming pipe and can route the mixture into the input pipe or into a bypass pipe.

5. The unit of claim 3 wherein the output box has a first output valve that can accept the mixture from the output pipe and/or from a bypass pipe and can route the mixture into an outgoing pipe.

6. The unit of claim 4 further comprising an output box having a plurality of valves for directing flows of the mixture.

7. The unit of claim 6 wherein the output box has a first output valve that can accept the mixture from the output pipe and/or from the bypass pipe and can route the mixture into an outgoing pipe.

8. The unit of claim 4 wherein the input box has a second input valve that can accept the mixture from a transmit pipe and/or from the first input valve and can route the mixture into a return pipe.

9. The unit of claim 5 wherein the output box has a second output valve that can accept the mixture from a station-bound pipe and/or from the first output valve and can route the mixture into a transmit pipe.

10. The unit of claim 8 further comprising an output box having a plurality of valves for directing flows of the mixture.

11. The unit of claim 10 wherein the output box has a first output valve that can accept the mixture from the output pipe and/or from the bypass pipe and can route the mixture into an outgoing pipe.

12. The unit of claim 11 wherein the output box has a second output valve that can accept the mixture from a station-bound pipe and/or from the first output valve and can route the mixture into a transmit pipe.

13. A modular algae culturing system, comprising:
(i) a plurality of units, each unit comprising:
(a) a substantially watertight main body comprising:
(1) a top plate substantially opposite a bottom plate;
(2) a back plate substantially opposite a front plate;
(3) an input side wall substantially opposite an output side wall;
(4) wherein the top plate, the bottom plate, the back plate, the front plate, the input side wall and the output side wall are placed in proximity to one another so as to define an interior space and an exterior space;
(5) a plurality of mixing members within the interior space, the plurality of mixing members being oriented generally horizontally;
(6) an input port providing access from the exterior space into the interior space; and
(7) an output port providing access from the interior space out to the exterior space;
(b) an input pipe wherein a mixture comprising at least a fluid can arrive from the input pipe and flow into the input port into the main body where the mixture can flow through the interior space and be mixed by the plurality of mixing members; and
(c) an output pipe wherein the mixture can flow out of the interior space via the output port and into the output pipe;
(ii) a plurality of processing stations;
(iii) a means for connecting the plurality of units and the plurality of processing stations.

14. The system of claim 13, each unit further comprising an input box having a plurality of valves for directing flows of the mixture.

15. The system of claim 13, each unit further comprising an output box having a plurality of valves for directing flows of the mixture.

16. The system of claim 14 wherein the means for connecting the plurality of units and the plurality of processing stations includes a plurality of pipes.

17. The system of claim 15 wherein the means for connecting the plurality of units and the plurality of processing stations includes a plurality of pipes.

18. The system of claim 16 wherein each of the plurality of processing stations includes at least a main feed tank and a means of moving the mixture through the system.

19. The system of claim 17 wherein each of the plurality of processing stations includes at least a main feed tank and a means of moving the mixture through the system.

20. The system of claim 18 wherein the means of moving the mixture through the system is a pump.

21. The system of claim 19 wherein the means of moving the mixture through the system is a pump.

22. A method of culturing algae using a modular algae culturing system, the method comprising:
configuring a modular algae culturing system;
introducing a mixture;
introducing a plurality of algae;
operating the modular algae culturing system;
bypassing a modular unit as needed;
harvesting a plurality of algae.

23. The method of claim 22 wherein the modular algae culturing system comprises:
(i) a plurality of units, each unit comprising:
(a) a substantially watertight main body comprising:
(1) a top plate substantially opposite a bottom plate;
(2) a back plate substantially opposite a front plate;
(3) an input side wall substantially opposite an output side wall;
(4) wherein the top plate, the bottom plate, the back plate, the front plate, the input side wall and the output side wall are placed in proximity to one another so as to define an interior space and an exterior space;
(5) a plurality of mixing members within the interior space, the plurality of mixing members being oriented generally horizontally;
(6) an input port providing access from the exterior space into the interior space; and
(7) an output port providing access from the interior space out to the exterior space;
(b) an input pipe wherein a mixture comprising at least a fluid can arrive from the input pipe and flow into the input port into the main body where the mixture can flow through the interior space and be mixed by the plurality of mixing members; and
(c) an output pipe wherein the mixture can flow out of the interior space via the output port and into the output pipe;
(ii) a plurality of processing stations;
(iii) a means for connecting the plurality of units and the plurality of processing stations.
